(19) United States
(12) Patent Application Publication
(10) Pub. No.: US 2014/0037725 A1
(43) Pub. Date: Feb. 6, 2014

(54) BILAYER PHARMACEUTICAL COMPOSITIONS OF NAPROXEN

(71) Applicant: CADILA HEALTHCARE LIMITED, Ahmedabad (IN)

(72) Inventors: Muthaiyyan Esakkimuthu KANNAN, Ahmedabad (IN); Gaurav Navinbhai MISTRY, Ahmedabad (IN); Priteshkumar Chadrakant MISTRY, Ahmedabad (IN)

(73) Assignee: CADILA HEALTHCARE LIMITED, Ahmedabad (IN)

(21) Appl. No.: 13/952,790

(22) Filed: Jul. 29, 2013

(30) Foreign Application Priority Data
Aug. 1, 2012 (IN) 2222/MUM/2012

(51) Int. Cl. A61K 9/24; A61K 31/192

(52) U.S. Cl. CPC A61K 9/209; A61K 31/192
USPC 424/465; 514/569; 264/241

(57) ABSTRACT

The present invention is related to a bilayer pharmaceutical composition comprising naproxen or a pharmaceutically acceptable salt thereof. The present invention is particularly related to a bilayer pharmaceutical composition comprising an immediate release component and a controlled release component of naproxen or a pharmaceutically acceptable salt thereof and the process for preparation thereof. Moreover, the present invention is related to the treatment of rheumatoid arthritis, oesteoarthritis, ankylosing spondylitis, pain, primary dysmenorrhoea, acute tendinitis, bursitis and acute gout.

### FIELD OF INVENTION

The present invention is related to a bilayer pharmaceutical composition comprising naproxen or a pharmaceutically acceptable salt thereof. The present invention is particularly related to a bilayer pharmaceutical composition comprising an immediate release component and a controlled release component of naproxen or a pharmaceutically acceptable salt thereof and the process for preparation thereof. Moreover, the present invention is related to the treatment of rheumatoid arthritis, osteoarthritis, ankylosing spondylitis, pain, primary dysmenorrhoea, acute tendinitis, bursitis and acute gout.

### BACKGROUND OF THE INVENTION

Naproxen is well known and widely used as anti-inflammatory, analgesic and antipyretic properties. It is chemically known as (+)-6-methoxy-alpha-methyl-2-naphthaleneacetic acid and has the following structure:

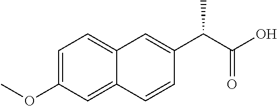 [SMILES: COc1ccc2cc([C@H](C)C(=O)O)ccc2c1]

Naproxen is used for the reduction of pain, fever, inflammation, and stiffness caused by conditions including migraine, osteoarthritis, kidney stones, rheumatoid arthritis, psoriatic arthritis, gout, ankylosing spondylitis, menstrual cramps, tendinitis, and bursitis. It is also used for the treatment of primary dysmenorrhea. The exact mechanisms of action have not been clearly established, but many of the effects of naproxen are associated with the inhibition of prostaglandin synthesis and in particular cyclooxygenase, an enzyme that catalyses the formation of prostaglandin precursors from arachidonic acid. Naproxen is used as either its free acid or its sodium salt, naproxen sodium.

Naproxen is available in the USA market in 250 mg, 375 mg and 500 mg dose as immediate release tablets and is generally administered in therapeutic doses of 500-1000 mg per day with dosing intervals of 8-12 hours (twice daily). The immediate release formulations have immediate absorption of drug and rapid onset of effect. However, the use of immediate release formulations requires more frequent dosing and is associated with frequent fluctuating plasma naproxen concentrations and thus produces toxic and undesired side effects.

Naproxen sodium is available in the USA market in 375 mg, 500 mg and 750 mg dose as extended release tablets and is generally administered in therapeutic doses of 550-1100 mg per day with dosing intervals of once daily. It is marketed under trade name of Naprelan® by Stat Trade Pharma.

U.S. Pat. No. 5,637,320 discloses a controlled release formulation of naproxen sodium for once a daily administration. It discloses once daily naproxen formulation comprising multi-particulate pellet form, each pellet having a core of naproxen and a multi-layer membrane surrounding the core. It describes the comparison of controlled release formulation of once a daily administration with commercially available twice daily naproxen immediate release tablet (Naprosyn®) and has shown superior efficacy and reduced adverse effects.

U.S. Pat. No. 4,803,079 discloses a controlled release tablet for once-daily oral administration of 500-1200 mg of naproxen or naproxen sodium comprises homogeneous matrix comprising: about 4-7 weight percent of hydroxypropyl methylcellulose and about 0.1 to about 2 weight percent of a pharmaceutically acceptable lubricating agent. It discloses that the low level of matrix material required by the present invention makes it possible to formulate once daily naproxen or naproxen sodium dosage form without excessive bulk, having weight and size characteristics which make it well-adapted for practical and acceptable patient administration.

U.S. Pat. No. 6,599,529 discloses two different portions of NSAID. The first portion is for quick release of NSAID and is made up of multiple units. The second portion is for controlled release of NSAID having multiple units and the release of NSAID is controlled by coating the multiple units with water-insoluble, but water-diffusible and substantially pH-independent polymers.

The prior art documents described above are related to a single unit controlled release compositions of naproxen as well as two different units compositions containing immediate release and controlled release of naproxen. Despite the availability of different types of compositions, it is cumbersome, inconvenient and expensive to manufacture compositions with separate components containing an immediate release and controlled release units of naproxen at commercial level. Moreover, there is a high probability in the fluctuation of plasma concentrations level of drug and occurrence of adverse effects using these types of compositions. Thus, there is a continuous unmet need to provide a composition, which is simple, stable and easy to manufacture and at the same time provides a steady state plasma concentration of drug.

The present invention is related to a bilayer pharmaceutical composition of naproxen or a pharmaceutically acceptable salt thereof containing two different components—an immediate release component and a controlled release component. The concept of bilayer tablet is generally employed for various purposes such as for the preparation of stable formulation, to achieve steady state plasma concentration of drug, ease in manufacture and economically compatible.

### SUMMARY OF THE INVENTION

The present invention in general aspect relates to a bilayer pharmaceutical composition comprising an immediate release component and a controlled release component of naproxen or a pharmaceutical acceptable salt thereof.

In one general aspect, there is provided a bilayer pharmaceutical composition comprises a) an immediate release component containing naproxen or a pharmaceutically acceptable salt thereof, one or more pharmaceutically acceptable excipients; and b) a controlled release component containing naproxen or a pharmaceutically acceptable salt thereof, one or more pharmaceutically acceptable rate-controlling substances and one or more pharmaceutically acceptable excipients.

In another general aspect, there is provided a bilayer pharmaceutical composition comprising naproxen or a pharmaceutically acceptable salt thereof one or more pharmaceutically acceptable excipients, wherein said pharmaceutically acceptable excipients are selected from a diluent, a binder, a disintegrant, a surfactant, a pH-regulating agent, a glidant, a lubricant or a combination thereof.

In another general aspect, there is provided a bilayer pharmaceutical composition comprising naproxen or a pharmaceutically acceptable salt thereof and one or more pharmaceutically acceptable rate-controlling substances, wherein said rate-controlling substances are selected from the group consisting of one or more of hydrophilic substance and/or a hydrophobic substance.

In another general aspect, there is provided a bilayer pharmaceutical composition comprising naproxen or a pharmaceutically acceptable salt thereof, wherein said composition provides an in-vitro release such that at least about 10% of naproxen is released after 1 hour, at least about 20% of naproxen is released after 2 hours, at least about 40% of naproxen is released after 4 hours, at least about 60% naproxen is released after 8 hours, and at least about 80% naproxen is released within 12 hours.

In another general aspect, there is provided a bilayer pharmaceutical composition comprising naproxen or a pharmaceutically acceptable salt thereof, wherein said composition exhibits an area under the curve (AUC₀₋ₜ) from about 500 μg/ml·hr to about 2500 μg/ml·hr; a maximum plasma concentration (Cₘₐₓ) from about 25 μg/ml to about 150 μg/ml; or time to achieve maximum concentration time (Tₘₐₓ) from about 2 hours to about 16 hours.

In another general aspect, there is provided a process of preparing a bilayer pharmaceutical composition comprising—

a) preparing an immediate release component by mixing and/or granulating naproxen or a pharmaceutically acceptable salt thereof with one or more pharmaceutically acceptable excipients;
b) preparing a controlled release component by mixing and/or granulating naproxen or a pharmaceutically acceptable salt thereof with one or more rate-controlling substances and one or more pharmaceutically acceptable excipients;
c) compressing the immediate release component and the controlled release component to form a bilayer composition.

In still another general aspect, there is provided a bilayer pharmaceutical composition of naproxen or a pharmaceutically acceptable salt thereof for the treatment of pain, fever, inflammation, and stiffness caused by conditions including migraine, osteoarthritis, kidney stones, rheumatoid arthritis, psoriatic arthritis, gout, ankylosing spondylitis, menstrual cramps, tendinitis, bursitis, and primary dysmenorrhea.

### DETAILED DESCRIPTION OF THE INVENTION

The present invention relates to a bilayer pharmaceutical composition comprising an immediate release component and a controlled release component of naproxen or a pharmaceutical acceptable salt thereof.

The inventors of the present invention have surprisingly found that it is possible to prepare stable bilayer pharmaceutical compositions of naproxen or a pharmaceutically acceptable salt thereof, which provides a steady-state plasma concentration with reduced side effects.

The inventors have further found that naproxen or a pharmaceutically acceptable salt thereof can be formulated into a bilayer composition comprising both an immediate release component and a controlled release component and that prevents peak-trough fluctuations in plasma levels and thus maintains therapeutically effective plasma levels of naproxen over an extended period of time.

In one embodiment, the bilayer pharmaceutical composition of the present invention comprises: a) an immediate release component containing naproxen or a pharmaceutically acceptable salt thereof, one or more pharmaceutically acceptable excipients; and b) a controlled release component containing naproxen or a pharmaceutically acceptable salt thereof, one or more pharmaceutically acceptable rate-controlling substances and one or more pharmaceutically acceptable excipients.

The term “naproxen” used throughout the specification refers to not only naproxen per se, but also its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof.

The term “pharmaceutically acceptable salts” refer to derivatives of the disclosed compound wherein the parent compound is modified by making acid or base salts thereof. Examples of pharmaceutically acceptable salts include, but are not limited to organic acid salts of basic residues such as amines, alkali or organic salts of acidic residues such as carboxylic acids, and the like. Preferably the present invention may comprise naproxen sodium.

The term “immediate release” shall mean that the release of the majority of the active material content occurs within a relatively short time, for example within 1 hour, preferably within 30 minutes, after oral ingestion. Immediate release can also be referred to as instant release.

The term “controlled release” refers to the release of a drug substance from a pharmaceutical formulation, at a slower rate than from an immediate release formulation. Controlled release can also be referred to as sustained release, prolonged release, extended release, or modified release.

The term “rate controlling substance” refers to a substance which can control the release of a drug from the dosage form for a prolonged period of time. The rate controlling substance may include a hydrophilic and/or a hydrophobic substance.

In one embodiment of the present invention, the bilayer pharmaceutical composition of naproxen or a pharmaceutically acceptable salt thereof, wherein the composition exhibits no significant difference in both rate and extent of absorption of naproxen or a pharmaceutically acceptable salt thereof as compared to immediate release formulation of naproxen marketed under trade name Naprosyn® administered twice daily.

In another embodiment, the bilayer pharmaceutical composition of the present invention is present in the form of a tablet.

The amount of naproxen or a pharmaceutically acceptable salt thereof employed in the composition of the present invention may vary according to the method of treatment in patients. It will be understood that the specific dose level for any particular patient will depend upon a variety of factors including body weight, gender, diet, time and route of administration, rate of absorption and excretion, and the severity of the particular disease being treated. Preferably, naproxen may be present in an amount from about 20% w/w to about 80% w/w, preferably from about 30% w/w to about 70% w/w, more preferably from about 40% w/w to about 60% w/w based on the total weight of composition.

The ratio of the amount of naproxen or a pharmaceutically acceptable salt thereof in an immediate release component to a controlled release component may be in the range of from about 1:2 to about 1:15.

The amount of naproxen that is incorporated in a tablet may range between about 250 mg and about 1500 mg. The tablet of the present invention provides a release period suitable for once-daily dosing, i.e. once within a hour period. Generally, naproxen and naproxen sodium are administered at levels of 375-750, 500-1000 or 750-1500 mg/day.

In another embodiment, the bilayer pharmaceutical composition comprises two distinct components having an immediate release and a controlled release of naproxen or a pharmaceutically acceptable salt thereof, wherein said components may be present in the form of powder, granules, pellets, seeds, minitablets, microtablets, spheres, beads, multiparticulates or a combination thereof.

In another embodiment, the bilayer pharmaceutical composition of the present invention comprises naproxen or a pharmaceutically acceptable salt thereof, and one or more pharmaceutically acceptable excipients, wherein said pharmaceutically acceptable excipients are selected from a diluent, a binder, a disintegrant, a surfactant, a pH-regulating agent, a glidant, a lubricant or a combination thereof.

Suitable diluents are selected from, but are not limited to microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like. The diluents or fillers may present in an amount from about 10% w/w to about 90% w/w, preferably from about 30% w/w to about 70% w/w, more preferably from about 20% w/w to about 50% w/w based on the total weight of composition.

Suitable binders are selected from, but are not limited to hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinylpyrrolidone, pregelatinized starch, sodium alginate, gums, synthetic resins or combination thereof. The binders may present in amount from about 1% w/w to about 25% w/w, preferably from about 10% w/w to about 20% w/w, more preferably from about 5% w/w to about 10% w/w based on the total weight of composition.

Suitable disintegrants are selected from, but are not limited to but not limited to starch or its derivatives, partially pregelatinized maize starch (Starch 1500), croscarmellose sodium, sodium starch glycolate, clays, celluloses, alginates (e.g. Kelton HVCR), pregelatinized corn starch, crospovidone (Kollidon CL-M), gums and the like used either alone or combination thereof. The disintegrants may present in amount from about 1% w/w to about 25% w/w, preferably from about 10% w/w to about 20% w/w, more preferably from about 5% w/w to about 10% w/w based on the total weight of composition.

Suitable surfactants are selected from, but are not limited to oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate and the like or combination thereof. The surfactant may present in amount from about 1% w/w to 10% w/w, preferably from about 4% w/w to about 8% w/w based on the total weight of the composition.

Suitable pH regulating agents are selected from, but are not limited to acidic substances, alkalizing agents, buffering agents or combination thereof. The acidic substances include, but are not limited to citric acid, malic acid, lactic acid, ascorbic acid, tartaric acid, fumaric acid, acetic acid, hydrochloric acid, sulphuric acid, gluconic acid, lactic acid, erythorbic acid, phosphoric acid, adipic acid, succinic acid, sorbic acid, formic acid or any combination thereof. The alkalizing agents include, but are not limited to sodium carbonate, potassium carbonate, sodium hydroxide, potassium hydroxide, calcium hydroxide, ammonium hydroxide, magnesium hydroxide, calcium oxide, magnesium oxide. Suitable buffering agents include, but are not limited to phosphate buffer, citrate buffer, trisodium phosphate, trisodium citrate, tripotassium phosphate. The pH regulating agents may present in an amount sufficient to maintain the pH of the composition in the required range.

Suitable lubricants and glidants are selected from, but are not limited to talc, metallic stearates such as magnesium stearate, calcium stearate, zinc stearate, colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; or combination thereof. The lubricants and glidants may present in amount from about 1% w/w to 5% w/w based on the total weight of composition.

The bilayer pharmaceutical composition of the present invention comprises one or more pharmaceutically acceptable rate-controlling substances, wherein said rate-controlling substances are selected from the group consisting of one or more of hydrophilic substances and/or hydrophobic substances.

Suitable hydrophilic substances are selected from, but not limited to one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polyalcohols, pectin, zein, modified zein, casein, gelatin, gluten, serum albumin, or collagen, chitosan, oligosaccharides and polysaccharides such as cellulose, dextrans, tamarind seed polysaccharide, gellan gum, carrageenan gum, xanthan gum, gum Arabic, hyaluronic acid, polyhyaluronic acid, alginic acid, sodium alginate acrylic acid or acrylamide derivatives and the like. The preferred hydrophilic substance is hydroxypropyl methylcellulose or any commercially available grade thereof such as methocel. Preferred hydrophilic substance is selected from hydroxypropyl cellulose, hydroxypropyl methylcellulose or mixture thereof.

The hydrophilic substances may present in an amount from about 10% w/w to about 50% w/w, preferably from about 20% w/w to about 40% w/w, or more preferably from about 25% w/w to about 30% w/w based on the total weight of the composition.

Suitable hydrophobic substances are selected from, but not limited to one or more of ethyl cellulose, glycerol palmitostearate, beeswax, glycowax, carnauba wax, hydrogenated vegetable oil and its derivatives, e.g. hydrogenated castor oil, glycerol monostearate, stearylalcohol, glyceryl behenate, polyanhydrides, acrylate and phthalate polymers and copolymers such as poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate) and the like. The hydrophobic substances may present in an amount from about 10% w/w to about 50% w/w, preferably from about 20% w/w to about 40% w/w, or more preferably from about 25% w/w to about 30% w/w based on the total weight of the composition.

In another embodiment, the bilayer pharmaceutical composition comprising naproxen or a pharmaceutically acceptable salt thereof exhibits an in vitro dissolution profile according to USP measurements using USP paddle apparatus (Apparatus-II) in phosphate buffer at pH 7.4 at 37° C. such that at least about 10% of naproxen is released after 1 hour, at least about 20% of naproxen is released after 2 hours, at least about 40% of naproxen is released after 4 hours, at least about 60% naproxen is released after 8 hours, and at least about 80% naproxen is released within 16 hours.

The bilayer pharmaceutical composition of the present invention exhibits no significant difference in the bioavailability of naproxen in a patient in terms of the fed state and fasting state bioavailability. The presence or absence of the food effect may be quantified by making Area under the Curve (AUC) and maximum plasma concentration (Cₘₐₓ) measurements according to methods known in the art. In quantitative terms, a pharmaceutical dosage form according to the present invention may be said to exhibit no food effect if the 90% confidence interval (CI) for the ratio of means of fed to fasted treatments fall within the interval of 80% to 125% of the AUC_{fed/fasting} and C_{max(fed/fasting)}. Thus, for example, the 90% confidence interval limits for the mean of the AUC in a patient between administrations under fed conditions must fall between 80 and 125% of the values obtained under fasting conditions.

In another embodiment, the bilayer pharmaceutical composition of the present invention comprising naproxen or a pharmaceutically acceptable salt thereof, wherein said composition exhibits a maximum plasma concentration (Cₘₐₓ) from about 25 μg/ml to about 150 μg/ml, preferably from about 50 μg/ml to about 125 μg/ml, more preferably from about 60 μg/ml to about 100 μg/ml.

In another embodiment, the bilayer pharmaceutical composition of the present invention comprising naproxen or a pharmaceutically acceptable salt thereof, wherein said composition exhibits an area under the curve (AUC₀₋ₜ) from about 500 μg/ml·hr to about 2500 μg/ml·hr, preferably from about 1000 μg/ml·hr to about 2000 μg/ml·hr, more preferably from about 1300 μg/ml·hr to about 1800 μg/ml·hr.

In still another embodiment, the bilayer pharmaceutical composition of the present invention comprising naproxen or a pharmaceutically acceptable salt thereof, wherein said composition exhibits time to achieve maximum concentration time (Tₘₐₓ) from about 2 hours to about 16 hours.

The bilayer pharmaceutical composition of the present invention may optionally comprise a non-functional coating.

The non-functional coating may comprise polymers like hydroxypropyl cellulose, hydroxyethyl cellulose, hydroxypropyl methylcellulose, polyvinylpyrrolidone, copolymer of vinyl pyrrolidone and vinyl acetate; plasticizers like polyethylene glycol, triacetin, dibutyl sebecate and diethyl tartrate; opacifying agents like titanium dioxide and talc; and coloring agents. Examples of such non-functional coat are commercially available Opadry compositions.

The process for preparation of bilayer pharmaceutical composition of the invention may involve wet granulation, dry granulation, direct compression, slugging, hot melt granulation, hot melt extrusion, fluidized bed granulation, extrusion-spheronization, spray drying and solvent evaporation. The wet granulation process involves use of water or any other suitable solvent. The dry granulation may involve use of roller compacter or any suitable technique.

In another embodiment, the bilayer pharmaceutical composition of the present invention may be prepared by the steps of:

a) preparing an immediate release component by mixing and/or granulating naproxen or a pharmaceutically acceptable salt thereof with one or more pharmaceutically acceptable excipients;
b) preparing a controlled release component by mixing and/or granulating naproxen or a pharmaceutically acceptable salt thereof with one or more rate-controlling substances and one or more pharmaceutically acceptable excipients;
c) compressing the controlled release blend and the immediate release blend using bilayer machine to form a bilayer compressed tablet.
d) optionally, film coating the bilayer tablet using film forming compositions.

The immediate release component of the present invention may be in the form of powder, granules, pellets, seeds, minitablets, microtablets, spheres, beads or multiparticulates and those may be prepared by direct mixing, wet granulation, dry granulation, extrusion spheronization technique as known by the skilled person in the art.

In another embodiment, the immediate release component of the present invention may be prepared by wet granulation method, wherein the method comprises the steps of blending naproxen or pharmaceutically acceptable salt thereof and one or more pharmaceutically acceptable excipients; granulating the blend with the help of aqueous or non-aqueous solution or dispersion of a binder to produce granules; drying the granules; mixing the dried granules with one or more pharmaceutically acceptable excipients to form the material ready to compress.

In another embodiment, the immediate release component of the present invention may be prepared by dry granulation method, wherein the method comprises the steps of blending naproxen or pharmaceutically acceptable salt thereof and one or more pharmaceutically acceptable excipients; roller compacting the blend to produce flakes; crushing the flakes to produce granules and extragranularly mixing the excipients to form the material ready to compress.

In another embodiment, the immediate release component of the present invention may be prepared by extrusion spheronization method; wherein the method comprises the steps of blending and granulating naproxen or pharmaceutically acceptable salt thereof and one or more pharmaceutically acceptable excipients; extruding and spheronizing the wet mass to produce the granules; mixing the dried granules with one or more pharmaceutically acceptable excipients to form the material ready to compress.

In another embodiment, the immediate release component of the present invention may be prepared by the method comprises the steps of providing an inert core of a pareil or non-pareil seeds, coating the inert core with aqueous or non-aqueous dispersion/solution of naproxen or pharmaceutically acceptable salt thereof and one or more pharmaceutically acceptable excipients; mixing the dried granules with one or more pharmaceutically acceptable excipients to form the material ready to compress.

The controlled release component of the present invention may be in the form of powder, granules, pellets, seeds, minitablets, microtablets, spheres, beads or multiparticulates and those may be prepared by direct mixing, wet granulation, dry granulation, extrusion spheronization technique as known by the skilled person in the art.

In another embodiment, the controlled release component of the present invention may be prepared by wet granulation method, wherein the method comprises the steps of blending naproxen or pharmaceutically acceptable salt thereof, one or more rate-controlling substances and one or more pharmaceutically acceptable excipients; granulating the blend with the help of aqueous or non-aqueous solution or dispersion of a binder to produce granules; drying the granules; mixing the dried granules with one or more pharmaceutically acceptable excipients to form the material ready to compress. The one or more rate-controlling substances may be added intragranularly or extragranularly. The granules may be individually coated with a functional coat having one or more rate-controlling substances.

In another embodiment, the controlled release component of the present invention may be prepared by dry granulation method, wherein the method comprises the steps of blending naproxen or pharmaceutically acceptable salt thereof, one or more rate-controlling substances and one or more pharmaceutically acceptable excipients; roller compacting the blend to produce flakes; crushing the flakes to produce granules and extragranularly mixing the excipients to form the material ready to compress. The one or more rate-controlling substances may be added intragranularly or extragranularly. The granules may be individually coated with a functional coat having one or more rate-controlling substances.

In another embodiment, the controlled release component of the present invention may be prepared by extrusion spheronization method; wherein the method comprises the steps of blending and granulating naproxen or pharmaceutically acceptable salt thereof, one or more rate-controlling substances and one or more pharmaceutically acceptable excipients; extruding and spheronizing the wet mass to produce the granules; mixing the dried granules with one or more pharmaceutically acceptable excipients to form the material ready to compress. The one or more rate-controlling substances may be added intragranularly or extragranularly. The granules may be individually coated with a functional coat having one or more rate-controlling substances.

In another embodiment, the controlled release component of the present invention may be prepared by the method comprises the steps of providing an inert core of a pareil or non-pareil seeds, coating the inert core with aqueous or non-aqueous dispersion/solution of naproxen or pharmaceutically acceptable salt thereof, optionally one or more rate-controlling substances and one or more pharmaceutically acceptable excipients; coating the drug layered cores with aqueous or non-aqueous dispersion/solution of one or more rate-controlling substances; mixing the coated material with one or more pharmaceutically acceptable excipients to form the material ready to compress.

In further embodiment, the present invention relates to a method of treatment of pain, fever, inflammation, and stiffness caused by conditions including migraine, osteoarthritis, kidney stones, rheumatoid arthritis, psoriatic arthritis, gout, ankylosing spondylitis, menstrual cramps, tendinitis, bursitis, and primary dysmenorrhea comprises administering the bilayer composition of the present invention to the patients in need thereof.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

### Bilayer Tablets of Naproxen Sodium

TABLE 1

| | Quantity per tablet (mg) | | |
|---|---|---|---|
| Ingredients | 750 mg | 500 mg | 375 mg |

a) Preparation of controlled release component of Naproxen Sodium

| | | | |
|---|---|---|---|
| Intragranular | | | |
| Naproxen sodium | 577.50 | 385.00 | 288.75 |
| Microcrystalline cellulose | 45.50 | 30.33 | 22.75 |
| Citric acid monohydrate | 30.00 | 20.00 | 15.00 |
| Hypromellose (Methocel K 4 M | 70.00 | 46.67 | 35.00 |
| Premium CR) | | | |
| Hypromellose (Methocel K100 | 45.00 | 30.00 | 22.50 |
| LVCR Premium) | | | |
| Granulation | | | |
| Purified water | q.s. | q.s. | q.s. |
| Extragranular | | | |
| Hypromellose (Methocel K 4 M | 50.00 | 33.33 | 25.00 |
| Premium CR) | | | |
| Hypromellose (Methocel K 15 M | 60.00 | 40.00 | 30.00 |
| Premium CR) | | | |
| Methacrylic acid Copolymer Type | 30.00 | 20.00 | 15.00 |
| A (Eudragit L 100) | | | |
| Talc | 30.00 | 20.00 | 15.00 |
| Lubrication | | | |
| Magnesium stearate | 10 | 6.67 | 5 |
| Total | 948 | 632 | 474 |

b) Preparation of immediate release component of Naproxen Sodium

| | | | |
|---|---|---|---|
| Intragranular | | | |
| Naproxen sodium | 247.50 | 165.00 | 123.75 |
| Microcrystalline cellulose | 147.50 | 98.33 | 73.75 |
| Granulation | | | |
| Povidone | 20.00 | 13.33 | 10 |
| Purified water | q.s | q.s | q.s |
| Extragranular | | | |
| Microcrystalline cellulose | 30.00 | 20.00 | 15.00 |
| Lubrication | | | |
| Magnesium stearate | 5 | 3.33 | 2.5 |
| Total | 450 | 300 | 225 |

c) Compression of controlled release and immediate release
components into bilayer tablet

Compression

| | | | |
|---|---|---|---|
| Controlled release blend | 948.00 | 632.00 | 474.00 |
| Immediate release blend | 450.00 | 300.00 | 225.00 |
| Total (Bilayer Core Tablet) | 1398 | 932 | 699 |
| Film Coating | | | |
| Opadry White 03B28796 | 42 | 28 | 21 |
| Purified Water | q.s | q.s | q.s |
| Total (Bilayer Coated Tablet) | 1440 | 960 | 720 |

Procedure:

### a) Preparation of Controlled Release Component of Naproxen Sodium

Naproxen sodium, microcrystalline cellulose, citric acid monohydrate, hypromellose (Methocel K4M Premium CR) and hypromellose (Methocel K100 LVCR Premium) were passed through sieve, were mixed together and granulated using purified water to yield wet granules. The wet granules were dried. These dried granules were mixed with hypromellose (Methocel K4M Premium CR), hypromellose (Methocel K15M Premium CR), methacrylic acid copolymer Type A (Eudragit L100) and talc. The granules were lubricated with magnesium stearate to yield final blend ready for compression.

### b) Preparation of Immediate Release Component of Naproxen Sodium

Naproxen sodium and microcrystalline cellulose were passed through sieve and were mixed together. Povidone was dispersed in purified water under constant stirring to yield the binder for granulation. The dry mixed blend of naproxen sodium was granulated using the binder solution of povidone to yield wet granules. The wet granules were dried and mixed with microcrystalline cellulose. The granules were lubricated with magnesium stearate to yield final blend ready for compression.
   c) Compression of Controlled Release and Immediate Release Components into Bilayer Tablets
The controlled release component and the immediate release components of naproxen sodium were compressed together using appropriate tooling to provide bilayer tablets.

### d) Coating

The compressed bilayer tablets were coated with coating dispersion of opadry to yield the finished product.

TABLE 2
Dissolution data of Example 1

| Time | % of naproxen |
|---|---|
| (hours) | released |
| 1 | 28 |
| 2 | 43 |
| 3 | 50 |
| 4 | 55 |
| 6 | 64 |
| 8 | 73 |
| 10 | 80 |
| 12 | 86 |
| 14 | 91 |

Dissolution Parameters:
Medium: phosphate buffer with pH 7.5
Volume: 900 ml
Apparatus: Paddle (USP apparatus Type II)
Revolution: 50 RPM

TABLE 3
Summary of PK parameters of Example 1 and reference
product (Naprelan ER Tablets)
Fasting stage:

| | Ratio | | | |
|---|---|---|---|---|
| Dependent | [% Ref] | CI_90_Lower | CI_90_Upper | Power |
| Ln(AUC_{INF}_{_{—}}_{obs}) | 101.53 | 97.54 | 105.68 | 1.0000 |
| Ln(AUCₗₐₛₜ) | 104.20 | 100.13 | 108.43 | 1.0000 |
| Ln(Cₘₐₓ) | 117.61 | 111.68 | 123.85 | 0.9999 |

Mean Tₘₐₓ of Ref. Product: 4.705 hr
Mean Tₘₐₓ of Test Product: 4.820 hr

Fed stage:

| | Ratio | | | |
|---|---|---|---|---|
| Dependent | [% Ref] | CI_90_Lower | CI_90_Upper | Power |
| Ln(AUC_{INF}_{_{—}}_{obs}) | 111.92 | 105.31 | 118.95 | 0.9997 |
| Ln(AUCₗₐₛₜ) | 106.02 | 103.08 | 109.04 | 1.0000 |
| Ln(Cₘₐₓ) | 91.37 | 85.30 | 97.87 | 0.9989 |

Mean Tₘₐₓ of Ref. Product: 7.556 hr
Mean Tₘₐₓ of Test Product: 14.528 hr

1. A bilayer pharmaceutical composition comprising a) an immediate release layer containing naproxen or a pharmaceutically acceptable salt thereof, one or more pharmaceutically acceptable excipients and b) a controlled release layer containing naproxen or a pharmaceutically acceptable salt thereof, one or more pharmaceutically acceptable rate-controlling substances and one or more pharmaceutically acceptable excipients.

2. The bilayer pharmaceutical composition according to claim 1, wherein the naproxen or a pharmaceutically acceptable salt thereof present in an amount in the range from about 100 mg to about 1500 mg.

3. The bilayer pharmaceutical composition according to claim 1, wherein the ratio of naproxen or a pharmaceutically acceptable salt thereof in immediate release layer to controlled release layer is in the range of from about 1:2 to about 1:15.

4. The bilayer pharmaceutical composition according to claim 1, wherein the pharmaceutically acceptable excipients are selected from a diluent, a binder, a disintegrant, a surfactant, a pH-regulating agent, a glidant, a lubricant or a combination thereof.

5. The bilayer pharmaceutical composition according to claim 1, wherein the one or more pharmaceutically acceptable rate-controlling substances are selected from the group consisting of a one or more of hydrophilic substances and/or hydrophobic substances.

6. The bilayer pharmaceutical composition according to claim 4, wherein the pH-regulating agent is selected from the group consisting of an acidifier, an alkalizing agent, a buffering agent or a combination thereof.

7. The bilayer pharmaceutical composition according to claim 4, wherein the immediate release layer comprising an acidifier is selected from the group consisting of citric acid, malic acid, lactic acid, ascorbic acid, tartaric acid, fumaric acid, acetic acid, hydrochloric acid, sulphuric acid, gluconic acid, lactic acid, erythorbic acid, phosphoric acid, adipic acid, succinic acid, sorbic acid, formic acid or combination thereof.

8. The bilayer pharmaceutical composition according to claim 5, wherein the hydrophilic substances are selected from one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polyalcohols, pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic, hyaluronic acid, polyhyaluronic acid, alginic acid, sodium alginate acrylic acid or acrylamide derivatives or combination thereof.

9. The bilayer pharmaceutical composition according to claim 5, wherein the hydrophobic substances are selected from one or more of ethyl cellulose, glycerol palmitostearate, beeswax, glycowax, carnauba wax, hydrogenated vegetable oil and its derivatives, e.g. hydrogenated castor oil, glycerol monostearate, stearylalcohol, glyceryl behenate, polyanhydrides, acrylate and phthalate polymers and copolymers such as poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate) or combination thereof.

10. The bilayer pharmaceutical composition according to claim 1, wherein the composition retains at least 80% of the potency of naproxen or salts thereof after storage for 3 months at 40° C./75% RH.

11. A bilayer pharmaceutical composition comprising naproxen or a pharmaceutically acceptable salt thereof, wherein the composition provides an in-vitro release such that at least about 10% of naproxen is released after 1 hour, at least about 20% of naproxen is released after 2 hours, at least about 40% of naproxen is released after 4 hours, at least about 60% naproxen is released after 8 hours, and at least about 80% naproxen is released within 12 hours.

12. The bilayer pharmaceutical composition according to claim 1, wherein said composition exhibits an area under the curve (AUC₀₋ₜ) from about 500 μg/ml·hr to about 2500 μg/ml·hr.

13. The bilayer pharmaceutical composition according to claim 1, wherein said composition exhibits a maximum plasma concentration (Cₘₐₓ) from about 25 μg/ml to about 150 μg/ml.

14. The bilayer pharmaceutical composition according to claim 1, wherein said composition exhibits a maximum concentration time (Tₘₐₓ) from about 2 hours to about 16 hours.

15. A process of preparing a bilayer pharmaceutical composition comprising—
a) preparing an immediate release component by mixing and/or granulating naproxen or a pharmaceutically acceptable salt thereof with one or more pharmaceutically acceptable excipients;
b) preparing a controlled release component by mixing and/or granulating naproxen or a pharmaceutically acceptable salt thereof with one or more rate-controlling substances and one or more pharmaceutically acceptable excipients;
c) compressing the immediate release component and the controlled release component to form a bilayer composition.

16. A method for treating pain, fever, inflammation, and stiffness caused by conditions including migraine, osteoarthritis, kidney stones, rheumatoid arthritis, psoriatic arthritis, gout, ankylosing spondylitis, menstrual cramps, tendinitis, bursitis, and primary dysmenorrhea wherein said method comprises administering a bilayer pharmaceutical composition of naproxen or a pharmaceutically acceptable salt thereof.
